(19) United States
(12) Patent Application Publication
(10) Pub. No.: US 2007/0208085 A1
(43) Pub. Date: Sep. 6, 2007

(54) Potent cytotoxicity and inhibition of pan-cell cycle progression by an alkylating anthraquinone

(75) Inventors: Agamemnon Antoniou Epenetos, London (GB); Klaus Pors, Saltaire (GB); Paul James Smith, Vale of Glamorgan (GB); Laurence H. Patterson, Bingley (GB)

Correspondence Address: DLA PIPER US LLP, 4365 EXECUTIVE DRIVE, SUITE 1100, SAN DIEGO, CA, 92121-2133, US

(73) Assignee: SOMANTA LIMITED, London (GB)

(21) Appl. No.: 11/655,043

(22) Filed: Jan. 17, 2007

Related U.S. Application Data
(60) Provisional application No. 60/759,693, filed on Jan. 17, 2006.

(51) Int. Cl. A61K 31/137

(52) U.S. Cl. 514/649

(57) ABSTRACT

The present invention generally relates to chemotherapeutic treatment of proliferative disorders, such as cancer. The invention more specifically relates to inhibition of pan-cell cycle progression with alkylating anthraquinones, which may inhibit mitotic commitment, lead to limited expression of G2 arrest and force cells to enter polyploidy via an aberrant mitosis. The methods of the invention are particularly useful in the treatment of chemotherapy-resistant cancers.

### RELATED APPLICATION DATA

This application claims priority under 35 U.S.C. 119(e) to U.S. Provisional Patent Application Ser. No. 60/759,693, filed Jan. 17, 2006, which is incorporated herein by reference in its entirety.

### FIELD OF THE INVENTION

The present invention generally concerns chemotherapeutic treatment of proliferative disorders such as cancer. The invention more specifically relates to inhibition of pan-cell cycle progression with alkylating anthraquinones.

### BACKGROUND OF THE INVENTION

A major problem with cancer chemotherapy is the emergence of drug resistance. Therefore, it is of considerable therapeutic interest to develop compounds that are able to interact with their cellular targets in ways that will circumvent resistance mechanisms.

Topoisomerases.

DNA topoisomerases are crucial to the maintenance of cancer cells in a proliferative state. Topoisomerase enzymes are involved in resolving topological problems in DNA, such as superhelical tension, that arise during most nuclear activities involving DNA. Topoisomerase II (topo II) catalyzes changes in DNA topology (between relaxed and supercoiled states) by transiently cleaving and re-ligating both strands of the double helix, whereas Topoisomerase I (topo I) acts by introducing one break in one strand of the DNA.

A number of clinically useful anti-tumor agents inhibit topoisomerase. For example, anthraquinone anti-tumor drugs such as mitoxantrone, are topo II inhibitors with proven success for the treatment of advanced breast cancer, non-Hodgkin's lymphoma, and acute leukemia.

Anticancer drugs that are DNA topoisomerase II inhibitors are cytotoxic because they reportedly form complexes with DNA and/or topoisomerase II enzymes. The complexes are thought to decrease the re-ligation rate, disrupt the cleavage-re-ligation equilibrium, and have a net effect of increasing cleavage. The increased cleavage can damage the DNA and lead to chromosomal breakage. Cells with irreparable DNA damage die by apoptosis.

Topo II is cell-cycle dependent and expression is higher in actively proliferating cells. Topo II inhibitors generally disrupt the cell-cycle during S phase because the increased concentration of DNA double-strand breaks interferes with DNA replication and triggers apoptosis.

Cytotoxic anthraquinones are thought to act by DNA intercalation. The slow rate of dissociation of these drugs is believed responsible for their potent cytotoxicity, the kinetics of which favours long-term trapping of topo-DNA complexes. However, currently available DNA intercalators at best promote a transient inhibition of topo II because topo-drug-DNA ternary complex can be reversed by removal of the intracellular drug pool.

There is thus a need for topo II poisons that are long-acting or irreversible, lead to persistent inhibition of this enzyme, evade conventional resistance mechanisms, and display long-term efficacy as cancer chemotherapeutics.

### BRIEF DESCRIPTION OF THE DRAWINGS

FIG. 1 is an illustration of synthetic Methods A and B.

FIG. 2 shows the formulae for monosubstituted Structure A, non-symmetrical Structure B and symmetrical Structure C.

### DESCRIPTION OF THE INVENTION

The synthesis of 1,4-disubstituted chloroethylaminoanthraquinones and their nonalkylating DNA-binding hydroxyethylaminoanthraquinone analogues has previously been described. See Pors et al., J. Med. Chem. 47:1856-1859. (2004) (attached hereto as Exhibit A, which is incorporated by reference in its entirety for any purpose); and Pors et al., Mol. Cancer Ther. 2:607-610 (2003) (attached hereto as Exhibit B, which is incorporated by reference in its entirety for any purpose); see also Epenetos, et al., Abstract submitted to 16^{th} International Prostate Cancer Update Meeting, Jan. 18-20, 2006 (attached hereto as Exhibit C, which is incorporated by reference in its entirety for any purpose). The compounds have the general structure of formulae I, II and III: 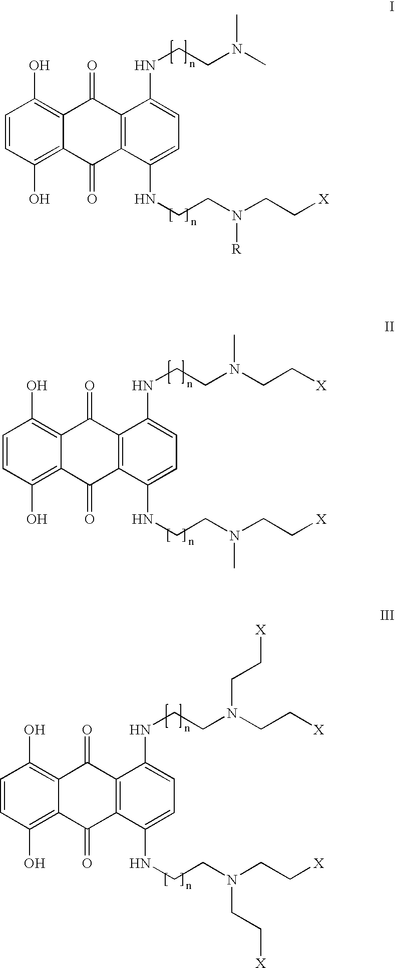
wherein:
R is H, CH₃, or (CH₂)ₙR′;
R′ is H, OH, halogen, SO₃CH₃, SO₃C₆H₅ or SO₃C₆H4CH₃;
X is halogen, SO₃CH₃, SO₃C₆H₅ or SO₃C₆H4CH₃; and
n is 1-6.

One compound in particular showed potent cytotoxicity against the A2780 human ovarian carcinoma cell line. The structure of this compound is given below as formula IV:

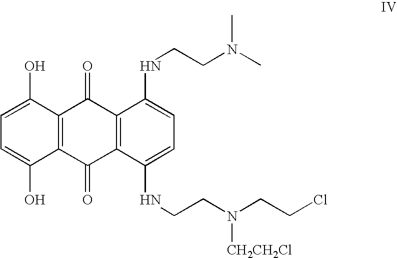

Previous studies demonstrated that covalently binding chloroethylaminoanthraquinones could be distinguished from noncovalently binding hydroxyethylaminoanthraquinones on the basis of cellular drug resistance. See Pors et al., 2003, supra. In particular, a covalently binding chloroethylaminoanthraquinone of formula IV demonstrated very low resistance, which was 20-150-fold less than that observed with the other chloroethylaminoanthraquinones tested and 250-fold less resistant than doxorubicin. Id. In addition in vitro studies showed that the compound of formula IV maintained potent cytotoxicity against doxorubicin-resistant and cisplatin-resistant tumor cell lines. Id.

The present invention provides methods for treating proliferative disorders, particularly cancer, using the compound of formula IV and related chloroethylaminoanthraquinones and hydroxyethylarninoanthraquinones. As illustrated in the EXAMPLES below, the compound of formula IV has been found effectively against a wide variety of tumor cells lines. Thus the cancer treated according to the methods of the present invention can be, without limitation, leukemia, melanoma, lung cancer, colon cancer, CNS cancer, ovarian cancer, renal cancer, prostate cancer, and breast cancer.

In one embodiment, compounds of the invention, particularly formula IV, display cytotoxicity toward proliferating cells. In one aspect, compounds of the invention slow or inhibit pan-cell cycle progression. In another embodiment of the invention, cells treated with compounds of the invention display limited expression of G2 arrest. Furthermore, some treated cells may become polyploid via an aberrant mitosis in order to escape G2 arrest.

The methods of the invention are particularly useful for treating proliferative disorders, such as cancer. The compounds of the invention display low resistance. In addition, compounds of the invention are cytotoxic in cells that are resistant to other chemotherapeutic agents including but not limited to cisplatin, doxorubicin, epirubicin, adriamycin and anthracycline. Thus, in one embodiment, compounds of the invention including formula IV, are used to treat chemotherapy-resistant proliferative disorders, such as chemotherapy-resistant cancer. The cancer can be resistant, for example, to cisplatin, doxorubicin, eprirubicin, adriamycin, anthracycline and other chemotherapy drugs that will be well known in the art.

The compound of formula IV is cytotoxic to cancer cells that display drug resistance due to a number of mechanisms. In one embodiment of the invention, methods are provided for treating chemotherapy resistant cells that show mismatch repair deficiency. In one aspect of this embodiment, the cancer has a defect in the expression of MLH1 protein. For example, cancer cells that are drug resistant due to under-expression or absence of MLH1 can be treated using methods of the present invention. In another embodiment of the invention methods are provided for treating chemotherapy resistant cells that show abnormal drug transport. For example, cancer cells that are drug resistant due to expression of P-glycoprotein (P-gp) can be treated using methods of the present invention.

As illustrated in the EXAMPLES below, compounds of the invention, particularly the compound of formula IV, slow or inhibit pan-cell cycle progression and mitotic commitment with a limited expression of G2 arrest. B1 cyclin tracking reveals that escape from cell cycle arrest in G2 induced by the compound of formula IV forces some cells to enter polyploidy via an aberrant mitosis, which is consistent with topoisomerase II inhibition.

Some of the mechanisms of cytotoxic compounds include altering the DNA thereby triggering the p53 pathway, or by affecting the enzymes which are involved in DNA replication and transcription. Cytotoxic compounds can also alter the cell cycle, for example, by causing arrest at one or more phases, or may ultimately cause apoptosis. The effects of such compounds can be analyzed using techniques such as flow cytometry or time-lapse imaging. The advantage of the time-lapse technique is that it records the images and events of individual cells.

In one embodiment of the present invention, the pharmacodynamics of the compound of formula IV has been investigated on a human osteosarcoma cell line (U-2 OS) with a functional tumor suppressor gene protein p53. This permitted monitoring of impact on cell cycle checkpoints for DNA damage. This novel approach uses time-lapse imaging of single cells expressing a cell cycle reporting green fluorescence protein (GFP) to track medium-term cellular responses. As detailed below in the Examples, the compound of formula IV slowed pan-cell cycle progression and mitotic commitment without any immediate induction of apoptosis. Cells treated with the compound exhibited a limited expression of G2 arrest. In addition, B1 cyclin tracking revealed that escape from cell cycle arrest in G2, which was induced by the compound of formula IV, forced some cells to enter polyploidy via an aberrant mitosis in keeping with topoisomerase II inhibition.

Thus according to the present invention, methods are provided for inhibiting or slowing pan-cell cycle progression in a cell comprising administering the compound of formula IV to the cell, thereby inhibiting or slowing pan-cell cycle progression of the cell. In another aspect of the invention, methods are provided for slowing mitotic commitment in a cell comprising administering the compound of formula IV to the cell. According to the methods of the invention, the treated cell may display limited expression of G2 arrest and some treated cells may become polyploid via an aberrant mitosis to escape the G2 arrest. In yet another aspect of the invention, treatment with the compound of formula IV inhibits topoisomerase II.

Having now generally described the invention, the same will be more readily understood through reference to the following examples (see also Exhibit C attached hereto, which is incorporated by reference in its entirety) which are provided by way of illustration, and are not intended to be limiting of the present invention, unless specified.

### EXAMPLES

### Example 1

### Synthesis of Chloroethylaminoanthraquinones

General Method for Synthesis of Anthraquinones

1-monosubstituted hydroxyethylamino-anthraquinones (HAQs) were prepared by the displacement of chloride from 1-chloroanthraquinone with an aminoalkylamino side chain in a polar solvent such as 2-methoxyethanol. The 1,4-disubstituted HAQs were synthesized as described in Pors et al. (J. Med. Chem., 47:1856-1859 (2004)) using previously described methods (Johnson et al., Cancer Treat. Rep., 63, 425-439 (1979); Murdock et al., J. Med. Chem. 1979, 22, 51-60) by the condensation of either leucoquinizarin or 5,8-dihydroxyleucoquinizarine (5,8,9,10-tetrahydroxyanthra-cene-1,4-dione) with an excess amount of N-alkyl-N-droxyalkylaminoalkylamine, which was synthesized as described in Aspinall (J. Am. Chem. Soc. 63: 852-853 (1948)) and Preston et al. (J. Med. Chem. 7:471-482 (1964). The 1,4-disubstituted HAQs were typically isolated at a yield between 20 and 50%, depending on the purity of the side chains used. Chlorination was subsequently achieved under mild conditions using triphenylphosphine-carbon tetrachloride complex (PPh₃-CCl₄). This method was highly efficient for the conversion of HAQs to chloroethyl derivatives, with approximately 80% yields.

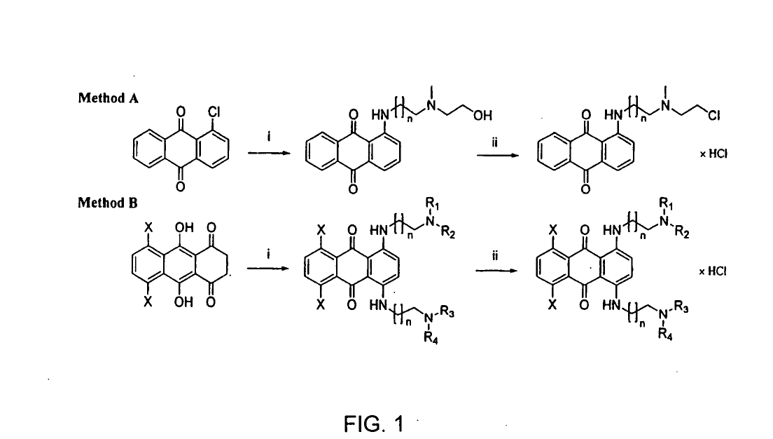

The general synthetic methods A and B of the invention follow the general schemes set forth in FIG. 1.

Method A: (i) N-alkyl-N-hydroxyalkylaminoalkylamine, reflux in 2-methoxyethanol for 6 h; (ii) Ph₃P—CCl₄ in CH₂Cl₂/CH₃CN (4:1) at room temperature for 48 hours, hydrochloride salt made by the addition of ethereal hydrogen chloride.

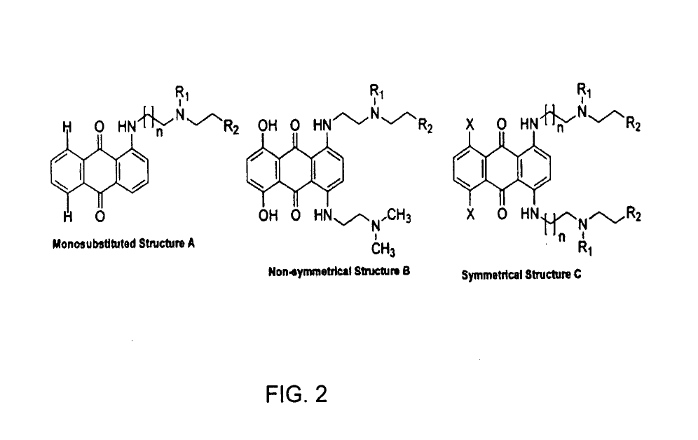

Method B: (i) leucoquinizarine (X═H) or 5,8-dihydroxyleucoquinizarine (X═OH) heated with appropriate N-alkyl-N-hydroxyalkylaminoalkylamine for 5 hours, R₁, R₂, R₃, R₄═CH₃ or CH₂CH₂OH, n=1 or 2); (ii) Ph₃P—CCl₄ in CH₂Cl₂/CH₃CN (4:1) at room temperature for 48 hours, hydrochloride salt made by the addition of ethereal hydrogen chloride, R₁ R₂, R₃, R₄═CH₃ or CH₂CH₂Cl, n=1 or 2). The compounds have the general formulae shown in Structures A, B, and C (FIG. 2) and are prepared as described in greater detail below. The constituents of certain specific compounds are given in Table 1, below.

TABLE 1

| | | | | |
|---|---|---|---|---|
| Structure | R₁ | R₂ | n | X |

Hydroxyethylaminoanthraquinone

| | | | | |
|---|---|---|---|---|
| 1 C | CH₂CH₂OH | OH | 1 | H |
| 2 C | CH₂CH₂OH | OH | 2 | H |
| 3 C | CH₃ | OH | 1 | H |
| 4 C | CH₂CH₂OH | OH | 2 | OH |
| 5 B | CH₃ | OH | — | OH |
| 6 C | CH₃ | OH | 1 | OH |
| 7 B | CH₂CH₂OH | OH | — | OH |
| 8 C | CH₂CH₂OH | OH | 1 | OH |
| 9 A | CH₃ | OH | 1 | — |
| 10 A | CH₃ | OH | 2 | — |

Chloroethylaminoanthraquinone

| | | | | |
|---|---|---|---|---|
| 11 C | CH₂CH₂Cl | Cl | 1 | OH |
| | CH₂CH₂Cl | | | |
| 12 B | CH₂CH₂Cl | Cl | — | OH |
| 13 C | CH₂CH₂Cl | Cl | 2 | OH |
| 14 A | CH₂CH₂Cl | Cl | 2 | — |
| 15 C | CH₃ | Cl | 1 | OH |
| 16 C | CH₃ | Cl | 1 | H |
| 17 A | CH₃ | Cl | 2 | — |
| 18 C | CH₂CH₂Cl | Cl | 2 | H |
| 19 C | CH₂CH₂Cl | Cl | 1 | H |
| 20 A | CH₃ | Cl | 1 | — |

### Synthesis of 1-[(2-[Dimethylamino]ethyl}amino]-4-[(2-[bis(2-hydroxyethyl)amino]ethyl}amino]-5,8-dihydroxyanthra-cene-9,10-dione

5,8-Dihydroxyleucoquinizarin (0.2 g, 0.75 mMol) was added to a mixture of N,N-bis(2-hydroxyethyl)-ethylenediamine (0.67 g, 3.6 mMol) and N,N-dimethylethyl-enediamine (0.194 g, 2.2 mmol) at 60° C. under N₂ and heated for 5 hours. The reaction was cooled to room temperature, and aq. NaOH (2 M, 0.2 cm³) was added and stirred overnight exposed to air. The reaction was diluted with CH₂Cl₂ (80 cm³), washed with water (3×80 cm³), dried with MgSO₄, and concentrated. The crude solid was purified by chromatography (CH₃OH:CH₂—Cl₂:NH₃, 4.5:94:0.5 increasing to 19.5:80:0.5). The resulting solid was further purified by redissolving in CH₃OH and precipitation with dry diethyl ether to give the product as dark blue solid (60.03 mg, 20% yield), mp 204.2-205° C.; ¹H NMR.

### 1-[{2-[Bis(2-chloroethyl)amino]ethyl}amino]-4-[{2-[di-methylamino]ethyl}amino]-5,8-dihydroxyanthracene-9,-10-dione dihydrochloride

Triphenylphosphine (0.11 g, 0.32 mmol) and then CCl₄ (0.19 g, 1.25 mmol) were added to a stirred solution of 7 (0.05 g, 0.10 mmol) in CH₂Cl₂ (5.0 cm³) under N₂. The resulting suspension was allowed to stir at room temperature for 24 hours. The residue was precipitated by the addition of dry ethereal HCl, isolated by filtration, and dried under vacuum. The crude product was dissolved in a minimum quantity of CH₂Cl₂/EtOH (1:1) at 60° C. and isolated from the triphenylphosphine oxide byproduct and excess triphenylphosphine by precipitation with EtOH/EtOAc (1:1). The product was isolated as a dark blue solid (51.5 mg, 83%). mp 190.0-192.1° C.; ¹H NMR (CDCl₃/CD₃OD) δ: 3.0 (s, 6H, 2×NCH3), 3.4 (t, 4H, 2×CH2N), 3.6 (t, 4H, 2×CH2N), 3.85 (t, 4H, 2×CH₂NHAr), 3.9 (t, 4H, 4×CH₂Cl), 7.1 (s, 2H, ArH), 7.3 (s, 2H, ArH), 10.6 (t, 2H, 2×NHAr); IR νₘₐₓ (KBr) cm⁻¹: 3600-3300 (OH), 1580 (C═O), 1230 (N—H); Eλ (CH₃OH/DMSO)=14162 cm⁻¹; νₘₐₓ (620 mn); FAB-MS, m/z(M+H)⁺509; Anal. (C₂₄H₃₀N₄O₄C₁₂.2HCl₂H₂O) C, H, N.

### Example 2

### Cell Cycle Events

In Vitro Studies

Cytotoxicity (IC₅₀) was determined using a 96-well plate-based 3-(4,5-dimethylthiazol-2-yl)-2,5-diphenyltetrazolium bromide assay with a 24-h drug exposure period and a 3-day growth period. Cells were maintained in RPMI 1640 containing glutamine (2 mM) and FCS (10%).

U-2 OS Cell line

The U2 OS cells were stably transfected with a Green Fluorescent Protein tagged Cyclin B1 reporter and maintained at 37° C. and 5% CO₂ in McCoys 5A modified medium (Sigma) supplemented with 2 mM glutamine, 100 units/ml penicillin, 100 μg/ml streptomycin, 10% fetal calf serum and 1000 μg/ml geneticin. Appropriate dilutions were added to the wells to obtain treatment regimes of 0, 10 and 100 nM of the experimental compound.

Timelapse Experimental Set-Up

The cells were plated in a 6 well plate (density ˜15-20%). Each experiment consisted of the paired drug treatment at the appropriate dose added to the cells and the plate was placed on a stage of an inverted microscope in a sealed environment (37° C./humidified 5% CO₂), and the entire apparatus was situated on a vibration-free table. Three fields were selected per well to triplicate the sampling for each experiment. Images at 10× magnification and were taken every 15 minutes over 3 days using a Cohu cooled CCD camera and the programme AQM Advance 6 to store the images. The images were viewed using the Metamorph programme.

Experimental data derived from the timelapse were analyzed by comparing the morphology of the cells and the relative growth rates per treatment regimen. This was achieved by counting the number of live cells per field every 12 hours (since a normal cell has a generation time of 24 hours) and plotting the data into relative growth curves. The initial rate of growth (between time points 0-12 and 12-24) was then calculated.

Flow Cytometry

Samples by flow cytometry. U-2 OS cells were treated with the compound in a 6 well plate in an incubator over 2 days. Cells were then extracted from the plate, and Draq5 added to stain the DNA before they were analyzed using a Becton Dickinson FACS Vantage flow cytometer. The flow data was gated for intact cells, and the DRAQ5 levels (indicator of DNA content) were extracted.

Results

DNA content and Cyclin B1 expression were measured using flow cytometry and a p53 functional human osteosarcoma cell line (U2-OS) as described above. Cells exposed to the compound of formula IV displayed slowed pan-cell cycle progression and mitotic commitment as compared to untreated cells, with a limited expression of G2 arrest, but without any immediate (<24 h) induction of apoptosis. In addition, B1 cyclin tracking revealed that escape from cell cycle arrest in G2, which was induced by the compound of formula IV, forced some cells to enter polyploidy via an aberrant mitosis in keeping with topoisomerase II inhibition.

These data indicate that the compound of formula IV has pan-cell cycle effects. The multilevel targeting by the compound of formula IV reduces the probability of evasion of cell cycle related pharmacodynamic responses. Furthermore these results help explain the activity of the compound of formula IV in both cisplatin and anthracycline resistant tumors in vitro and in vivo.

### Example 3

### In Vitro Activity of the Compound of Formula IV

To identify the activity of the compound of formula IV in additional cell lines, an NCI human cell line panel was screened for sensitivity to the compound. Cytotoxicity was evaluated for chloroethylaminoanthraquinone compounds synthesized as described in EXAMPLE 1, using an in vitro cell based assay as described in Pors et al., Mol. Cancer Ther., 2:607-610 (2003).

Human tumor cell lines from the NCI human cell line panel (Table 2) were tested in the assay.

TABLE 2
Cytotoxicity of Compound IV in Human Cell Lines

| | Type of | | | | |
|---|---|---|---|---|---|
| | Cell Line | Cell Line | Log IC₅₀ | IC₅₀ | IC₅₀ nM |
| 1 | Leuk | RPMI 8226 | −8 | <1 × 10⁻⁸* | <10* |
| 2 | NSCLC | A549/ATCC | −7.57 | 2.6915 × 10⁻⁸ | 27 |
| 3 | | EKVX | −6.12 | 7.5858 × 10⁻⁷ | 759 |
| 4 | | NCI-H226 | −7.27 | 5.3703 × 10⁻⁸ | 54 |
| 5 | | NCI-H322M | −6.53 | 2.9512 × 10⁻⁷ | 295 |
| 6 | | NCI-H460 | −8 | <1 × 10⁻⁸* | <10* |
| 7 | Colon | HCT-116 | −8 | <1 × 10⁻⁸* | <10* |
| 8 | | HCT-15 | −8 | <1 × 10⁻⁸* | <10* |
| 9 | | HT-29 | −8 | <1 × 10⁻⁸* | <10* |
| 10 | | KM 12 | −6.86 | 1.3804 × 10⁻⁷ | 138 |
| 11 | CNS | SF 268 | −8 | <1 × 10⁻⁸* | <10* |
| 12 | | SF 539 | −8 | <1 × 10⁻⁸* | <10* |
| 13 | | U251 | −7.72 | 1.9055 × 10⁻⁸ | 19 |
| 14 | Melanoma | LOX IMVI | −8 | <1 × 10⁻⁸* | <10* |
| 15 | | SK MEL 5 | −7.15 | 7.0795 × 10⁻⁸ | 71 |
| 16 | | UACC-257 | −5.73 | 1.8621 × 10⁻⁶ | 1862 |
| 17 | | UACC-62 | −6.66 | 2.1878 × 10⁻⁷ | 219 |
| 18 | Renal | 786-0 | −8 | <1 × 10⁻⁸* | <10* |
| 19 | | ACHN | −8 | <1 × 10⁻⁸* | <10* |
| 20 | | SN12C | −7.57 | 2.6915 × 10⁻⁸ | 27 |
| 21 | | TK10 | −6.39 | 4.0738 × 10⁻⁷ | 407 |
| 22 | Prostate | PC3 | −7.3 | 5.0119 × 10⁻⁸ | 50 |
| 23 | | DU145 | −7.3 | 5.0119 × 10⁻⁸ | 50 |
| 24 | Breast | MCF7 | −8 | <1 × 10⁻⁸* | <10* |
| 25 | | MDA-MB- | −7 | 1 × 10⁻⁷ | 100 |
| | | 231/ATCC | | | |
| 26 | | HS 578T | −6.28 | 5.2481 × 10⁻⁷ | 525 |
| 27 | | MDA-MB- | −7.68 | 2.0893 × 10⁻⁸ | 21 |
| | | 435 | | | |
| 28 | | BT549 | −5.69 | 2.0417 × 10⁻⁶ | 2042 |

*Value reported for IC₅₀ is less than 10 nm. Actual IC₅₀ was not reached in assay.

Growth inhibition was determined, and was expressed as the concentration (nM) required to inhibit cell growth by 50% (IG₅₀). The compound of formula IV was found to be potently active with a mean IC₅₀ of 49 nM against the NCI human cell lines, which includes several prostate cancer cell lines. In particular, excellent activity was observed against CNS, colon, NSCLC and renal cancer cell lines. Eleven of 28 cell lines tested had an IC₅₀ of <10 nM. Standard methods were used for MTT results (Monks A et. al., J. Natl Cancer Inst. 1991 June 5;83(11):757-66).

These data indicate that the compound of formula IV possesses potent activity across a variety of different human tumors including prostate cancer. In addition, the compound of formula IV showed potent activity in cisplatin and anthracyline resistant human tumors.

What is claimed is:

1. A method for slowing or inhibiting pan-cell cycle progression in a cell comprising administering the compound of formula IV: 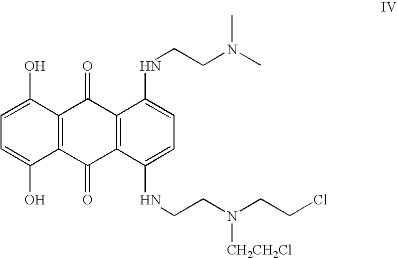
to the cell, thereby slowing or inhibiting pan-cell cycle progression the cell.

2. A method for slowing or inhibiting mitotic commitment in a cell comprising administering the compound of formula IV: 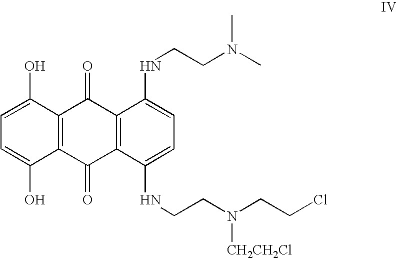
to the cell, thereby slowing or inhibiting mitotic commitment in the cell.

3. The method of claim 2, wherein the cell displays limited expression of G2 arrest.

4. The method of claim 2, wherein the compound inhibits mitotic commitment.

5. The method of claim 3, wherein some treated cells become polyploid via an aberrant mitosis to escape the G2 arrest.

6. The method of claim 4, wherein administering the compound of formula IV inhibits topoisomerase II.
